(19) United States
(12) Patent Application Publication
(10) Pub. No.: US 2023/0109784 A1
(43) Pub. Date: Apr. 13, 2023

(54) VENTILATOR AND VENTILATOR VALVE

(71) Applicant: University of Vermont and State Agricultural College, Burlington, VT (US)

(72) Inventor: Jason H.T. Bates, S. Burlington, VT (US)

(21) Appl. No.: 17/906,973

(22) PCT Filed: Mar. 26, 2021
(86) PCT No.: PCT/US2021/024537

§ 371 Date: Sep. 21, 2022

Related U.S. Application Data
(60) Provisional application No. 63/000,237, filed on Mar. 26, 2020.

(51) Int. Cl. A61M 16/20; A61M 16/10; A61M 16/08

(52) U.S. Cl. CPC A61M 16/201; A61M 16/1065; A61M 16/0833; A61M 16/209

(57) ABSTRACT

In a first embodiment, a ventilator has a housing with a first fixed port and a rotatable shutter configured to cooperate with the housing to enclose an interior of the housing. The shutter has a first orifice at a first radial distance from its axis of rotation. A stationary plate abuts the shutter and includes a first stationary orifice configured to at least partially align with the first orifice of the shutter over a first rotational distance of the shutter. In this way, the first orifice and the first stationary orifice form a first variable port. The stationary plate may have a second stationary orifice and the shutter may have a second orifice configured to at least partially align with the second stationary orifice over a second rotational distance of the shutter.

### CROSS-REFERENCE TO RELATED APPLICATIONS

This application claims priority to U.S. Provisional Application No. 63/000,237, filed on Mar. 26, 2020, now pending, the disclosure of which is incorporated herein by reference.

### FIELD OF THE DISCLOSURE

The present disclosure relates to devices for providing breathing gas to individuals, and more particularly, to ventilators.

### BACKGROUND OF THE DISCLOSURE

In a hospital and other medical settings, individuals may need the assistance of a ventilator when they cannot breathe on their own. Ventilators are expensive machines, and consequently hospitals tend not to have a large number of excess ventilators. Pandemics are relatively infrequent, but potentially devastating mass casualty events. In the event of a pandemic, such as the pandemic caused by the SARS-CoV-2 virus, the number of individuals that need ventilators may exceed the available supply in certain locales. Accordingly, there is a need for inexpensive ventilators that can be easily manufactured and operated.

### BRIEF SUMMARY OF THE DISCLOSURE

In an embodiment, a ventilator is provided. The ventilator has a housing with a first fixed port. A shutter is configured to cooperate with the housing to enclose an interior of the housing. The shutter is rotatable about an axis of rotation. The shutter has a first orifice at a first radial distance from the axis of rotation. A stationary plate abuts the shutter. The stationary plate has a first stationary orifice. The first orifice of the shutter is configured to at least partially align with the first stationary orifice of the stationary plate over a first rotational distance of the shutter. In this way, the first orifice and the first stationary orifice form a first variable port. In some embodiments, the shutter is contained within the housing. In some embodiments, the shutter is configured to rotate reciprocally between two angular positions.

The stationary plate may have a second stationary orifice and the shutter may have a second orifice configured to at least partially align with the second stationary orifice over a second rotational distance of the shutter. In this way, the second orifice and the second stationary orifice form a second variable port. The second orifice may be at a second radial distance from the axis of rotation. The second radial distance may be greater than, less than, or equal to the first radial distance.

The housing may include a separator configured to divide the interior of the housing into an inner chamber and an outer chamber. The first fixed port and the first variable port may be located on the inner chamber, and the second fixed port and the second variable port may be located on the outer chamber.

In some embodiments, an adjustment plate abuts the stationary plate. The adjust plate has a first adjustment slot configured to at least partially align with the first stationary orifice of the stationary plate. The adjustment plate may further include a second adjustment slot configured to at least partially align with the second stationary orifice of the stationary plate. The adjustment plate may be configured to be rotated to vary an alignment of the first adjustment slot with the first stationary orifice. Each of the first adjustment slot and the second adjustment slot may further include a wiper configured to extend through the respective first or second stationary orifice of the stationary plate to contact the shutter.

In some embodiments, the ventilator includes a first duct disposed on the adjustment plate and sized to interface with the first adjustment slot of the adjustment plate. In some embodiments, a motor is configured to rotate the shutter relative to the housing. In some embodiments, the ventilator further includes a crank configured to rotate the shutter when the crank is turned by an operator.

In another aspect, the present disclosure provides a ventilator valve. The ventilator valve includes a housing with an inlet for receiving breathing gas. The housing may have a cover with an outlet in fluid communication with the inlet. A shutter is rotatably disposed in a path between the inlet and the outlet. The shutter has an orifice configured to be in intermittent alignment with the inlet and the outlet. In this way, as the shutter is rotated, the inlet and outlet are occluded when the orifice is not aligned (where the shutter blocks the gas pathway between the inlet and the outlet) and patent when the orifice is at least partially aligned between the inlet and the outlet (where the orifice of the shutter allows gas flow in the gas pathway between the inlet and the outlet). In some embodiments, the ventilator valve includes an exhaust pressure regulator for controlling a positive-end-of-expiration pressure (“PEEP”) connected to the outlet of the housing. In some embodiments, the shutter is a disk or a cylinder.

In another aspect, the present disclosure may be embodied as a ventilator having a Y-shaped conduit. The Y-shaped conduit has an inspiratory leg configured to be connected to a source of breathing gas, a patient leg configured to be connected to an endotracheal tube, and an expiratory leg. A safety valve may be coupled to the inspiratory leg. The ventilator includes a valve according to any of the devices described herein. The ventilator may include an inspiratory demand valve connected to the inspiratory leg. The ventilator may include a filter coupled in the expiratory leg such that gas flowing through the expiratory leg is filtered. The ventilator may have a second ventilator valve according to any of the valves described herein. The second ventilator valve may be connected to the inspiratory leg and configured to be synchronized with the first ventilator valve such that gas flow through the inspiratory leg is out of phase with gas flow through the expiratory leg.

### DESCRIPTION OF THE DRAWINGS

For a fuller understanding of the nature and objects of the disclosure, reference should be made to the following detailed description taken in conjunction with the accompanying drawings, in which:

FIG. 1 shows two plots of ventilation parameters over time during APRV: pressure (top) and flow (bottom);

FIG. 2 shows two plots of ventilation parameters over time during conventional pressure-controlled ventilation: pressure (top) and flow (bottom);

FIG. 3 is an exploded view diagram showing a ventilator according to an embodiment of the present disclosure;

FIG. 4 is a cross-sectional diagram of the ventilator of FIG. 3;

FIGS. 5A-5C depict an exemplary shutter (5A), an exemplary stationary plate (5B), and an exemplary adjustment plate (5C), where the set is configured for conventional pressure-support ventilation;

FIGS. 6A-6B show the set of shutter and plates of FIGS. 5A-5C in an exploded view (6A) and stacked together (6B);

FIGS. 7A-7B shows an exemplary disk set for APRV, where (7A) is the stationary plate, and (7B) is the adjustment plate;

FIG. 8A is a diagram showing an adjustment plate configured to occlude the right end of the stationary orifice of the stationary plate, and the orifice of the shutter is coincident with the same right end of the portion of the stationary orifice. It can be seen that gas is able to pass through the variable port because of the thickness of the stationary plate.

FIG. 8B shows where a barrier of the adjustment slot extends through the stationary orifice to prevent gas escaping via the pathway depicted by the arrow.

FIG. 9 is a diagram showing a duct in communication with an adjustment slot of an adjustment plate;

FIG. 10 is a portion of a ventilator according to another embodiment;

FIGS. 11A and 11B depict the ventilator portion of FIG. 10 in an inspiratory position and an expiratory position;

FIG. 12 is a diagram depicting the operation of a reciprocating ventilator according to another embodiment of the present disclosure;

FIG. 13 is a diagram showing the driver of the ventilator of FIG. 12;

FIG. 14 is an exploded view schematic of a reciprocating ventilator;

FIG. 15 is a diagram of a reciprocating shutter;

FIG. 16 shows a geometric construction describing the calculation of overlap between a trip bar and a catch bar of a reciprocating ventilator;

FIG. 17 is another ventilator according to an embodiment of the present disclosure;

FIG. 18 is a view of the ventilator of FIG. 17 showing a driver;

FIG. 19 is an exploded view of a ventilator valve according to the present disclosure;

FIG. 20 is a diagram of a ventilator used in a patient setting; and

FIG. 21 is a schematic of a Vermontilator embodiment.

### DETAILED DESCRIPTION OF THE DISCLOSURE

Airway Pressure Release Ventilation (APRV) is a mode of ventilation that has shown remarkable efficacy in acute lung injury. It was recently used successfully in the intensive care unit (ICU) at the University of Vermont Medical Center (UVMMC) to ventilate an individual with COVID-19. The specific type of APRV used at UVMMC has been called “time-controlled adaptive ventilation” (TCAV). In this form of APRV, expiration is terminated when expiratory flow has fallen to a set fraction, α, of peak expiratory flow. Nieman et al. have shown efficacy when α=0.75, but also conversely that enormous lung damage can be caused if α=0.25. In other words, expiration must be brief—typically 0.5 s or less. Research at UVM Lamer College of Medicine (LCOM) over the past decade leads to the hypothesis that TCAV is protective because it never allows enough time during expiration for epithelial surfaces to come into apposition, meaning they never have to be pulled apart again during inspiration. This eliminates atelectrauma, which appears to be the central culprit in causing ventilator-induced lung injury (VILI), as opposed to tissue over-distention which is the commonly held view.

APRV is defined by the following parameters:

P_{high} constant pressure during inspiration

P_{low} constant pressure during expiration

T_{high} time during which high inspiratory pressure is applied

T_{low} time during which low expiratory pressure is applied

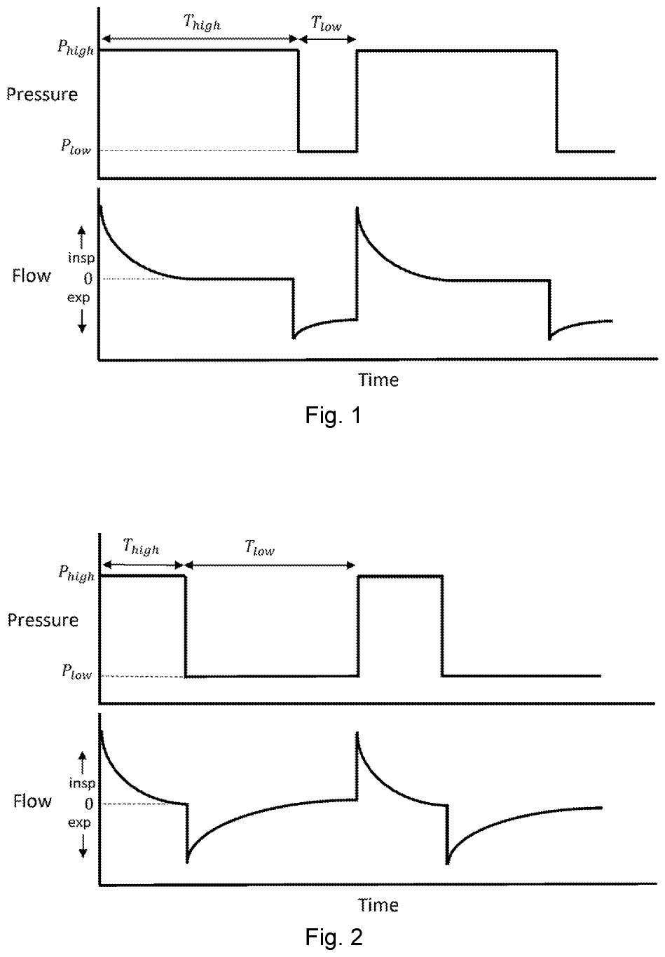

The pressure and flow profiles that occur during APRV are shown schematically in FIG. 1. FIG. 2 depicts pressure and flow profiles during conventional pressure-controlled ventilation.

Based on the above, it may be advantageous to provide protective ventilation in severely ill individuals (for example, individuals with COVID-19) using APRV that retains the essential features of TCAV, including an expiratory duration (sometimes called “release time”) that is on the order of 0.5 s. The present disclosure may be embodied as a ventilator that can be configured to provide such ventilation in a simple, inexpensive manner.

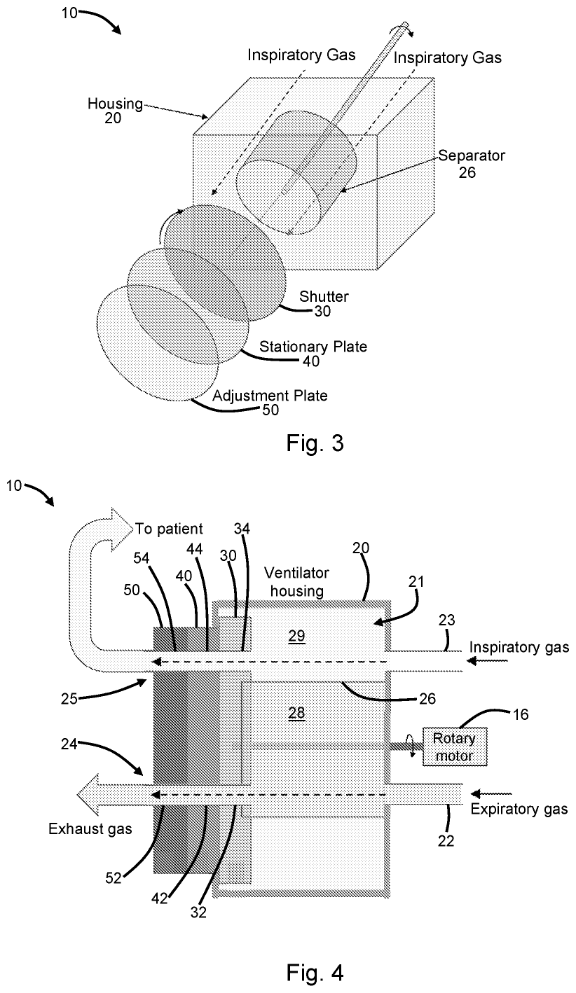

With reference to FIGS. 3 and 4, in a first aspect, the present disclosure may be embodied as a ventilator 10 for providing breathing gas to an individual. The ventilator 10 includes a housing 20 with a first fixed port 22. The first fixed port 22 may be an inlet, such as an inlet for receiving fresh breathing gas (inspiration) from a gas source. In other embodiments, the first fixed port 22 may be an outlet, such as an outlet for exhausting breathing gas (expiration).

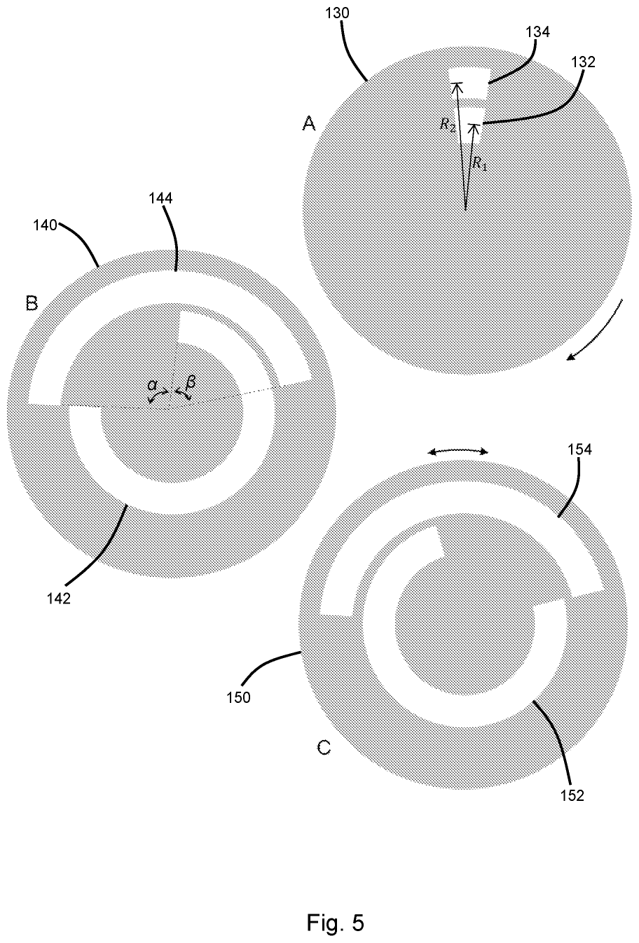

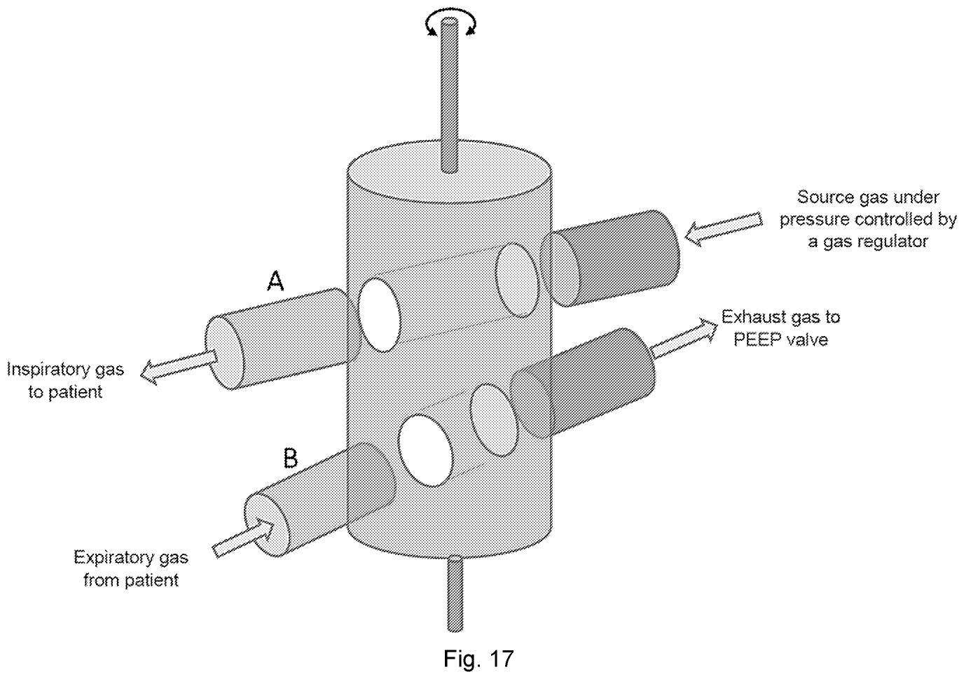

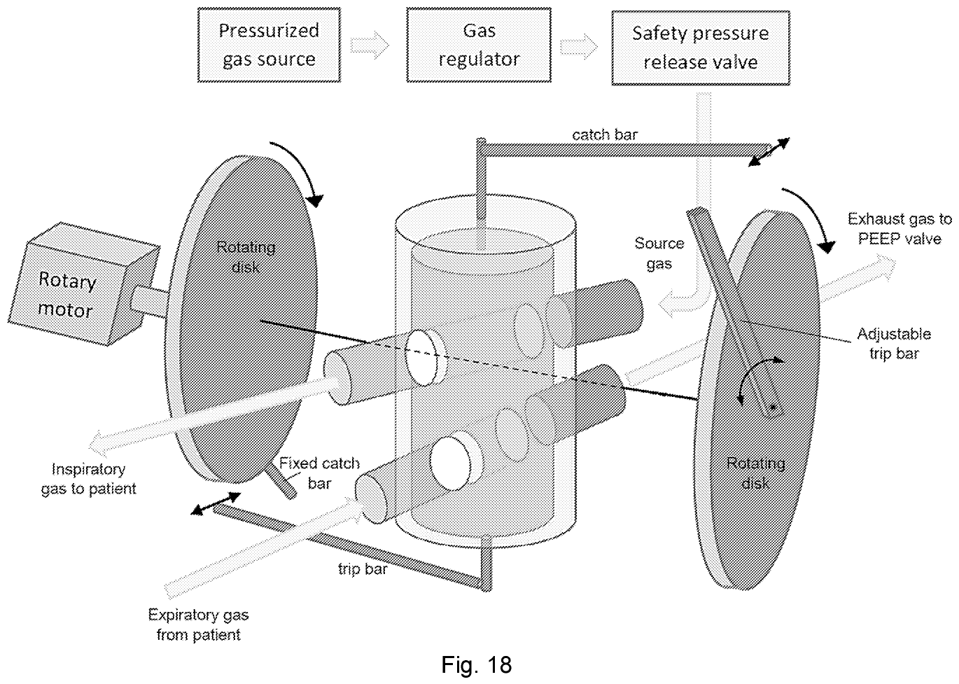

A shutter 30 cooperates with the housing 20 to enclose an interior 21 of the housing. The shutter may be shaped as a disk, such as the shutter 30 depicted in FIG. 3. The shutter may abut an open end of a housing. In some embodiments, the shutter is contained within the housing. In such a configuration, the shutter may abut a lip of the housing. The shutter is configured to be rotatable about an axis of rotation. For example, in a disk-shaped shutter, the axis of rotation may be at the center of the disk. The shutter 30 includes a first orifice 32. The first orifice is located at a first radial distance from the axis of rotation. The first orifice together with the first fixed port cooperate to form a gas flow path through the housing. In embodiments where the shutter is contained within the housing, it may be advantageous to configure the first fixed port as an inlet such that the gas flow path will urge the shutter into an improved sealing relationship with the housing. Performance of the device 10 generally benefits from improved control of gas flow (e.g., prevention of leaks, etc.) FIG. 5A depicts a shutter 130 having a first orifice 132 at a first radial distance, R₁, from the axis of rotation. In some embodiments, the shutter may be shaped as a cylinder (see, e.g., FIGS. 17 and 18) and the center of rotation may be the primary longitudinal axis of the cylinder shape.

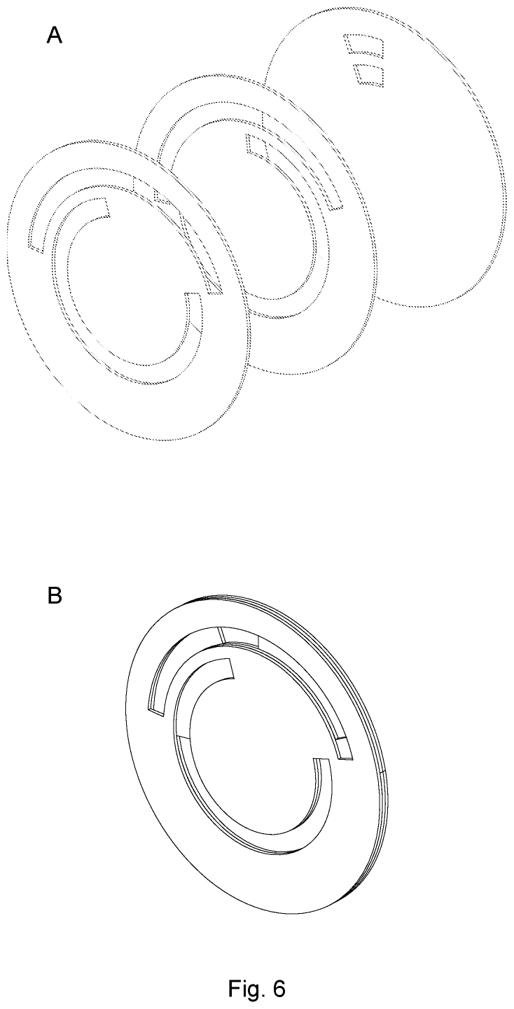

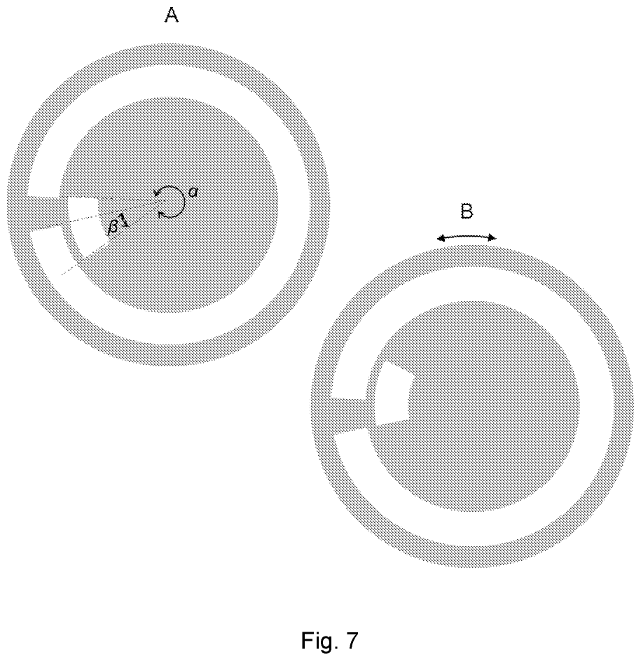

A stationary plate 40 abuts the shutter 30. The stationary plate 40 includes a first stationary orifice 42. The first stationary orifice 42 is configured such that as the shutter 30 rotates about the axis of rotation, the first orifice 32 moves into and out of alignment (at least partial alignment) with the first stationary orifice 42 (see also FIGS. 6A and 6B). As such, the first orifice 32 and the first stationary orifice 42 can be considered to work together as a first variable port 24. A gas flow path between the first fixed port 22 and the first variable port 24 may be occluded by rotating the shutter until the first orifice 32 is not aligned with the first stationary orifice 42. The first orifice 32 is configured to at least partially align with the first stationary orifice over a first rotational distance of the shutter 30. Rotational distance (i.e., angular displacement) may be measured as the distance in degrees of rotation of the shutter during which the first orifice 32 is at least partially aligned (coincident) with the first stationary orifice 42. FIG. 5B shows a stationary plate 140 having a first stationary orifice 142.

In some embodiments, such as that depicted in FIG. 3, the stationary plate 40 includes a second stationary orifice 44, and the shutter 30 includes a second orifice 34. The second orifice of the shutter may be located at a second radial distance, R₂, from the axis of rotation (see, e.g., second orifice 134 in FIG. 5A). The second radial distance R₂ may be greater than the first radial distance R₁. The second orifice 34 is configured to at least partially align with the second stationary orifice 44 over a second rotational distance of the shutter. In this way a second variable port 25 may be formed. The housing 20 may have a separator 26 configured to divide the interior 21 of the housing 20 into an inner chamber 28 and an outer chamber 29. The first fixed port and the first variable port may be located on the inner chamber 28. A second fixed port 23 and the second variable port 25 may be located on the outer chamber 29. In the exemplary ventilator of FIG. 3 (having a rotating disk-shaped shutter), the separator 26 may be configured as a cylindrical separator having a radius that is R₁<Rₛₑₚₐᵣₐₜₒᵣ<R₂.

It should be noted that the first radial distance and the second radial distance may be measured in any manner. For example, the first radial distance may be measured along a radius to a center of the first orifice (as shown in FIG. 5A). In another example, the first radial distance may be measured to a radially furthest location of the first orifice, or a radially shortest distance to a location of the first orifice. The second radial distance may be measured in the same way or in a different way as the first radial distance. Appropriate measurements for a particular application will be apparent to one having skill in the art in light of the present disclosure.

By including two gas flow paths, both inspiration and expiration can be regulated by the ventilator. Using a second gas flow path allows for (1) cyclically opening and closing the inspiratory gas pathway alternately with the expiratory pathway, (2) allowing the inspiratory pressure to be adjusted, and (3) providing adjustability of the inspiratory/expiratory duty cycle.

In some embodiments, an adjustment plate is provided to allow adjustment of the first and second variable ports without reconfiguration/replacement of the shutter or the stationary plate. For example, an adjustment plate may be configured to block a portion of the first stationary orifice. Such an adjustment plate 50 may abut the stationary plate 40 and include a first adjustment slot 52. Such a first adjustment slot 52 is configured to at least partially align with the first stationary orifice 42 of the stationary plate 40. The adjustment plate 50 is configured to be moved so as to change the alignment of the first adjustment slot and the first stationary orifice. In this way, the size of the variable port (maximum size of the first variable port) may be changed by moving the adjustment plate relative to the stationary plate, even while the ventilator is in operation (e.g., while the shutter is rotating to ventilate a patient). In other embodiments, the adjustment plate may be otherwise moved into a position so as to change the size of the variable port (translated, rotated about a position other than the center, etc.) For example, in the exemplary ventilator 10 of FIG. 3, the adjustment plate 50 may be rotated relative to the stationary plate 40 in order to alter the alignment between the first stationary orifice 42 and the first adjustment slot 52.

The adjustment plate may be configured to block a portion of second stationary orifice. For example, adjustment plate 50 may include a second adjustment slot 54 configured to at least partially align with the second stationary orifice 42 of the stationary plate 40. In some embodiments, a second adjustment plate may be included to change the size of the variable port (by blocking a portion of the second stationary orifice).

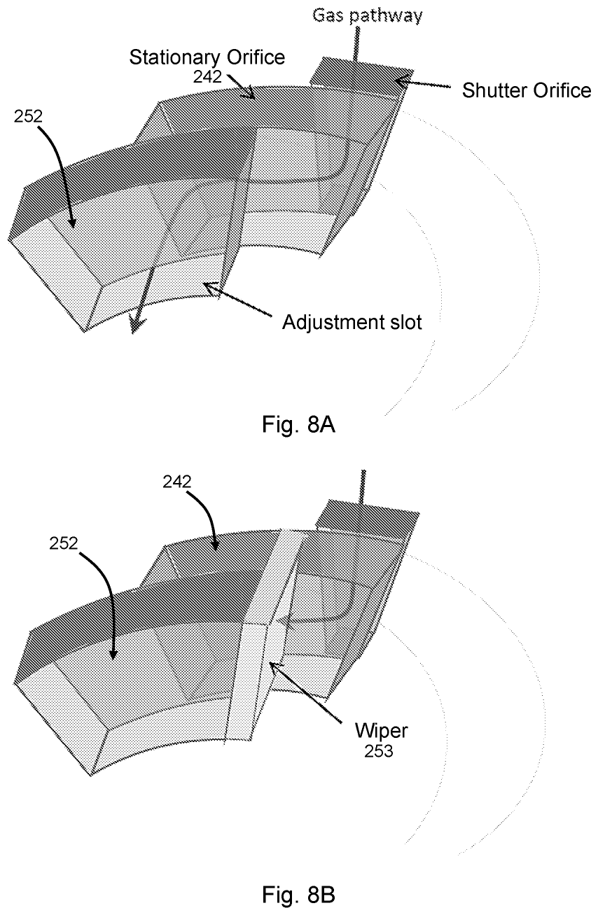

Because the stationary plate has a finite thickness, it will be possible for gas to escape through the variable port even where the first stationary orifice is partially covered by the adjustment plate despite the stationary orifice being ostensibly covered by the adjustment plate (illustrated in FIG. 8A). To prevent such escape of gas, the slots 252 of the adjustment plate may each include a wiper 253 configured to extend through the first stationary orifice 242 to contact the shutter (see FIG. 8B). Note that in FIGS. 8A and 8B, the shaded volumes indicate orifices and slots (with the exception of the wiper 253 of FIG. 8B).

FIGS. 5A-5C, 6A, and 6B show an exemplary set of plates for a ventilator according to the present disclosure, including a shutter (5A), a stationary plate (5B), and an adjustment plate (5C). Each of the members of the depicted set is designed to work cooperatively with the others to provide traditional pressure support ventilation in which the duration of expiration is longer than that of inspiration. As the shutter rotates, its two orifices move in to and out of alignment with the two corresponding stationary orifices of the stationary plate so as to alternately open the inspiratory and expiratory pathways. The two stationary orifices of the stationary plate are arranged concentrically such that the outer slot subtends an angle α+β from the axis of rotation (in this case, the center of the disk), while the inner slot subtends an angle 2π−α. One end of the radially-inner (first) stationary orifice and the associated end of the radially-outer (second) stationary orifice lie along a radial line, while the other two ends are at different radial positions, as shown in FIG. 5B.

The slots in the stationary plate are the same size as those in the adjustment plate except that the edges of the stationary orifices are at opposite ends as those of the corresponding slots (e.g., the first stationary orifice and the first slot) are at opposite ends (FIG. 5C vs FIG. 5B). The adjustment plate can be rotated through an angle β allowing for variable portions of the first and second stationary orifice to be occluded. The degree of occlusion is adjustable through an angle α that allows for a corresponding degree of adjustment of the inspiration: expiration duty cycle. This mirror-image arrangement of the slots of the adjustment plate from the stationary orifices of the stationary plate means that for every angular position of the adjustment plate there is no radial overlap between the inspiratory and expiratory pathways.

The rotation period of the shutter is T. Because of the geometry of the concentric slots in the stationary plate (FIG. 5B), the duration of inspiration lies in the interval

$$[ \frac{α T}{2 π} , \frac{( α + β ) T}{2 π} ] ,$$

while the duration of expiration lies in the interval

$$[ \frac{( 2 π - α ) T}{2 π} , \frac{( 2 π - α - β ) T}{2 π} ] .$$

The configuration of the adjustment plate disk may be such that, for any orientation of the adjustment plate, the sum of the inspiratory and expiratory durations is T. The adjustment plate can be rotated through angle β, in order to contravary the durations of inspiration and expiration. The degree of adjustability of the inspiration: expiration duty cycle is subject to the geometric constraint α>β.

FIGS. 7A and 7B show a stationary plate (7A) and an adjustment plate (7B) suitable for use with APRV ventilation. A ventilator configured for pressure support ventilation (using, for example, the plates in FIGS. 5A and 5B) can be reconfigured for APRV by, for example, exchanging the stationary and adjustment plates.

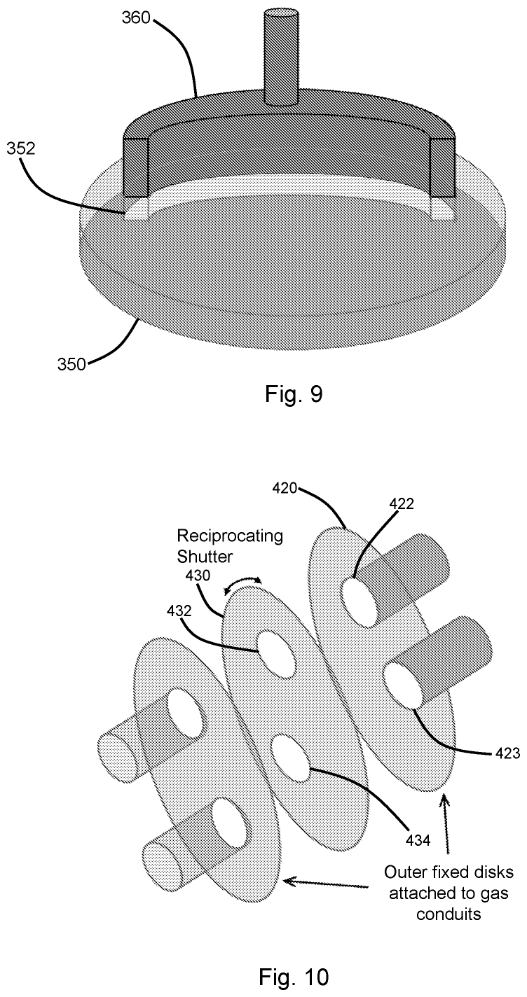

The ventilator may further include a first duct 360 configured to be in fluid communication with the first adjustment slot 352 of the adjustment plate 350 (see FIG. 9). The first duct may abut the adjustment plate along the perimeter of the first adjustment slot. In this way, gas passing through the first variable port (e.g., first orifice, first stationary orifice, and first adjustment slot) will be routed through the duct (e.g., plenum created by the duct) as desired. For example, the gas may be routed through the duct and into a conduit. In embodiments without an adjustment plate, the first duct may be configured to be in fluid communication with the first stationary slot of the stationary plate.

The ventilator 10 may further include a motor 16 coupled to the shutter 30. The motor may be configured to rotate the shutter. The motor may be any type of motor known in the art. For example, an electric motor, a pneumatic motor, etc. The motor may be directly coupled to the shutter, for example, by way of an axle sealed through the housing drives. The motor may drive the shutter at a constant angular velocity. The rotational period determines the respiratory rate (breaths per minute). In some embodiments, the valve includes a crank (or a coupler for connection to a crank) for manual rotation of the shutter. In this way, the shutter may be rotated “by hand” or other manual operation. Such a crank or coupler may be present in addition to or as an alternative to a motor.

The ventilator may further include a pressure regulator on the first port or the second port (whichever is configured to provide inspiratory gas flow) allowing the inspiratory pressure to vary between appropriate limits. The ventilator may further include an exhaust pressure regulator on a duct receiving the expiratory gas flow in order to provide a positive end-of-expiration pressure (PEEP).

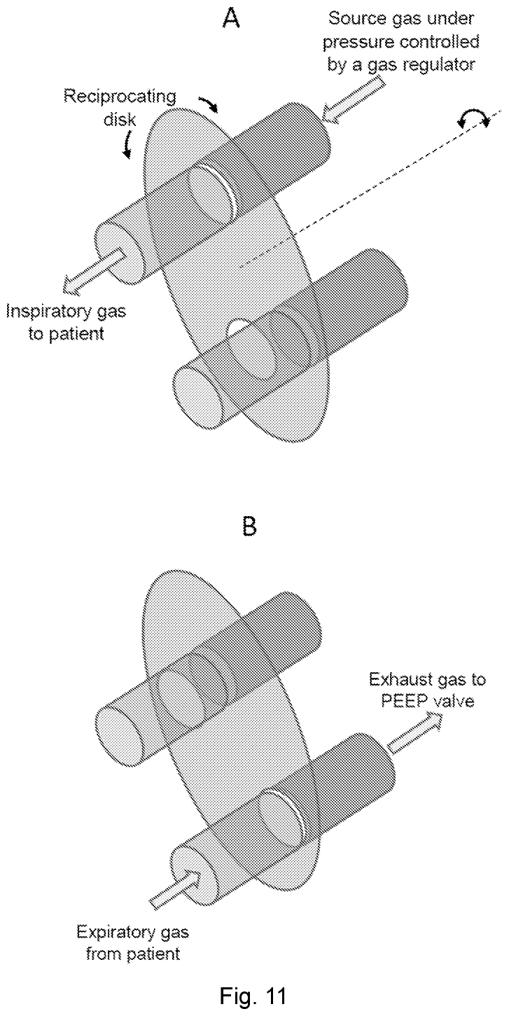

With reference to FIGS. 10-18, in some embodiments, the shutter is configured to rotate reciprocally between two angular positions. For example, a ventilator may include a shutter 430 having a first orifice 432. The shutter may further include a second orifice 434. The shutter 430 may abut a housing 420 having a first fixed port 422. In the case of the ventilator partially depicted in FIG. 10, the shutter 430 further includes a second orifice 434 and the housing 420 further includes a second fixed port 423. The shutter rotates back and forth (i.e., reciprocates) between (1) a first position where the first orifice at least partially aligns with the first fixed port and the second orifice blocks the second fixed port; and (2) a second position where the first orifice blocks the first fixed port and the second orifice at least partially aligns with the second fixed port. FIG. 11 shows an embodiment whereby the inspiratory and expiratory gas pathways alternate in patency; only one can be open at any given time. The inspiratory/expiratory gas conduits are bisected by a reciprocating shutter sandwiched between housings. The housings are attached to the two halves of the two conduits. The shutter rotates flush against the housings. The shutter has two orifices with diameters which may be equal to the inner diameters of the conduits (fixed ports). The shutter rotates both clockwise and counterclockwise between two extreme positions. In one position, a first one of the orifices of the shutter is coincident with the lumen of the inspiratory gas conduit; this allows inspiratory gas to flow while the expiratory conduit is closed. In the other position of the shutter, a second one of the orifices of the shutter is coincident with the lumen of the expiratory gas conduit; this allows expiratory gas to flow while the inspiratory conduit is closed. FIG. 10 shows an exploded view of shutter and housings.

Note that a reciprocating embodiment may be configured with a single orifice of the shutter moving between inspiratory and expiratory positions. In such an embodiment, a “dead zone” between the positions where the orifice is not coincident with either gas conduit may be used in order to avoid mixing inspiratory and expiratory gases. The use of two orifices avoids this; as one orifice moves out of alignment with one pathway, the other orifice simultaneously moves into alignment with the other pathway. This will require less angular movement of the reciprocating disk. Some embodiments using two orifices also have the advantage of not being vulnerable to complete closure of the breathing circuit in the event that a single orifice becomes stuck in the transition position.

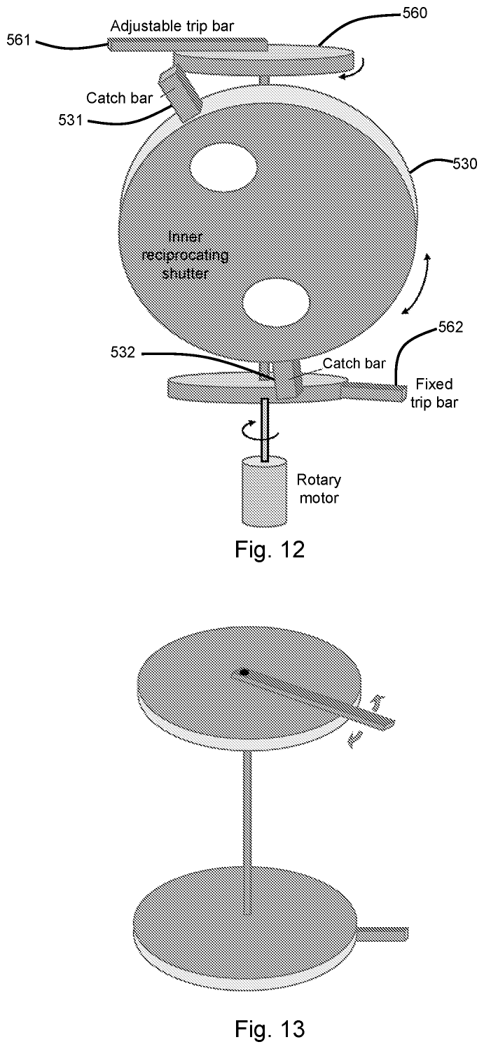

FIG. 12 shows a shutter 530 and an exemplary driver 560 to reciprocate the shutter. In this exemplary embodiment, the shutter 530 includes a first catch bar 531 and a second catch bar 532. The driver 560 includes a first rotating trip bar 561 and a second rotating trip bar 562. The first and second rotating trip bars may be rotated by a motor or the like. The first rotating trip bar 561 is aligned such that it will engage the first catch bar 531 at a first location during its rotation and move the first catch bar to a second location where it will disengage the first catch bar. The first catch bar 551 is arranged on the shutter 530 such that its movement (caused by engagement with the first trip bar 561) will rotate the shutter. Similarly, the second rotating trip bar 561 is aligned such that it will engage the second catch bar 531 at a first location during its rotation and move the second catch bar to a second location where it will disengage the second catch bar. The second catch bar 551 is arranged on the shutter 530 such that its movement (caused by engagement with the second trip bar 561) will rotate the shutter in a direction opposite the rotation caused by the first catch bar, thereby returning the shutter to its original rotational position. In this way the shutter is caused to rotate reciprocally—moved in a first rotational direction by the first catch bar and in a second rotation direction by the second catch bar.

FIG. 13 shows how the durations of expiration and inspiration may be adjusted. Since the durations of inspiration and expiration add up to the total breath period, their respective values are determined by the angular position of the first catch bar relative to that of the second catch bar. Adjustability is achieved by making the orientation of one or both catch bar adjustable.

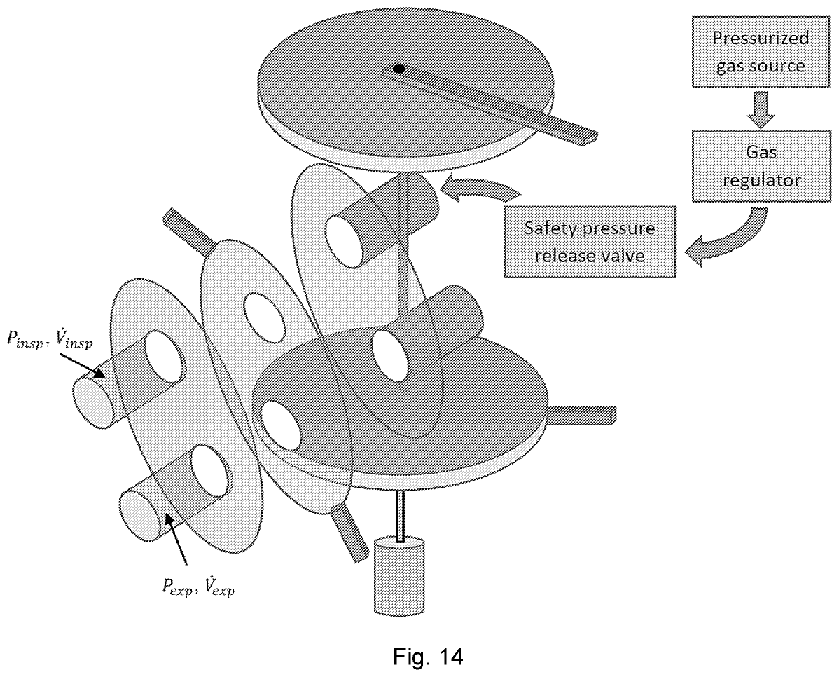

FIG. 14 shows an exploded view of an exemplary ventilator incorporating the reciprocating shutter and driver depicted in FIGS. 10-13. One or more of the depicted components may be housed in a case with entry and exit ports for inspiratory and expiratory gases, and a lid that can be opened to expose the position-adjustable first trip bar.

The following discussion details two design parameters that may be defined for the above-described reciprocating embodiment to work within the desired parameter ranges:
(1) The size and placement of the two orifices in the reciprocating shutter. This determines the angular displacement that the shutter must rotate through in order to switch between inspiration and expiration, as well as the change in the vertical positions of the ends of the two catch bars.
(2) The positions of the two trip bars which push on the catch bars until the latter move off of the plane of the corresponding trip bar.

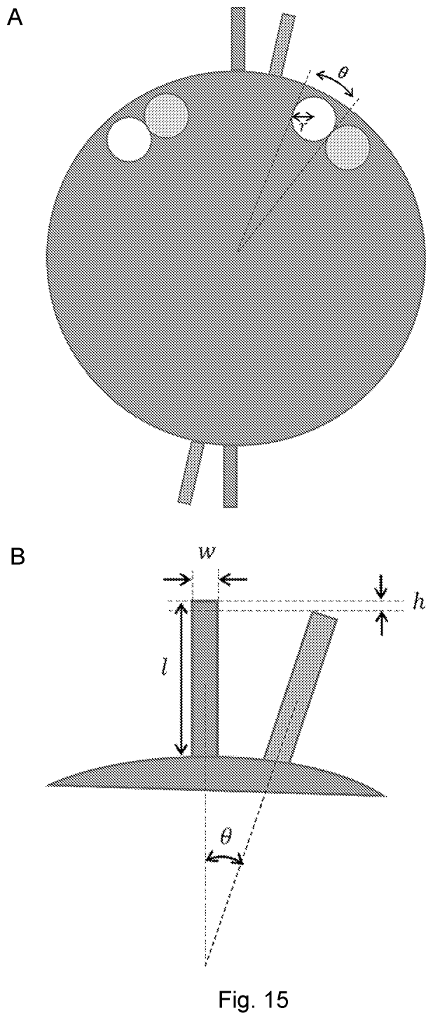

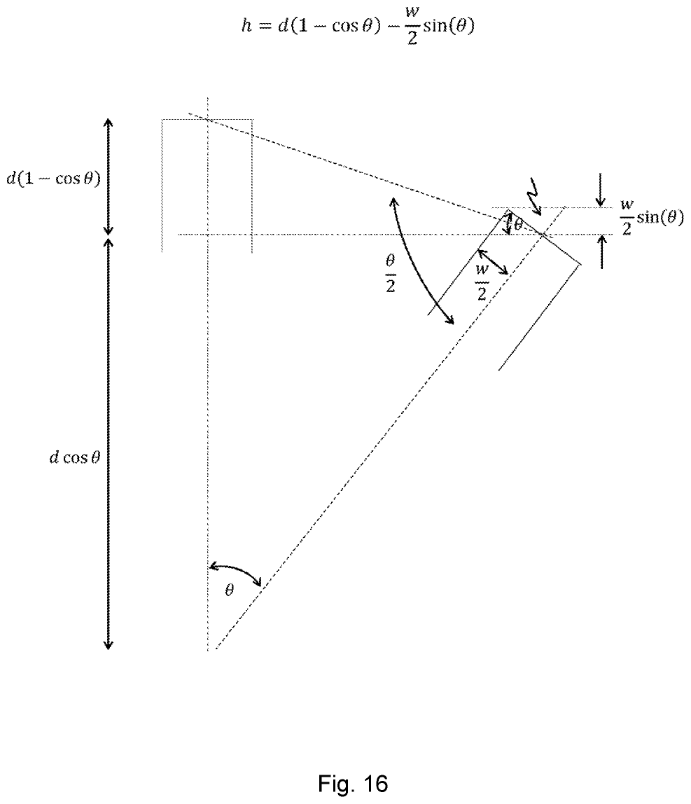

The relevant dimensions of the reciprocating shutter and its two orifices are shown in FIG. 15. The catch bars on the shutter have width w and length 1. When a catch bar is vertical it overlaps with the position of the associated trip bar by an amount h. As the trip bar pushes on the catch bar it rotates the shutter through an angle θ before the two bars (trip bar and catch bar) lose contact with each other. The value of h depends on the distance d from the rotational axis of the shutter to the tip of the catch bar, the width w of the catch bar, and the angle θ that the shutter must move in order to close one gas pathway and open the other one. FIG. 16 shows the geometric construction leading to the formula:

$$h = d ( 1 - cos θ ) - \frac{w}{2} sin ( θ ) \qquad ( 1 )$$

The angular separation of the upper and lower trip bars determines the fractions of the breath that corresponds to inspiration versus expiration. The shortest separation possible between switching is given by the angle subtended by one of the orifices (θ) as a fraction of 2π radians times the rotation period. If the ratio of inspiration to expiration can be as much as 10:1 (as can be the case in APRV) then 9 is 36 degrees. Suppose, for example, that the radius of the shutter out to the end of the catch bar is 5 cm, and that the catch bar itself has a width of 0.5 cm. The above formula gives:

h=5(1−cos 36)−0.25 sin(36)=0.81 cm (2)

In another embodiment using a rotating shutter, the shutter is configured as a rotating cylinder. FIG. 17 illustrates components of an exemplary the cylindrical shutter that switches between inspiratory and expiratory gas pathways. FIG. 18 shows the reciprocating cylindrical shutter contained with a housing. Using a similar driver as for the reciprocating shutter of FIG. 14, the rotating cylinder assembly may be incorporated into an orthogonal unidirectional rotating assembly incorporating catch bars and trip bars, as illustrated in FIG. 18.

With reference to FIG. 18, the present disclosure may be embodied as a ventilator valve 600 for a ventilator for providing breathing gas to an individual. The ventilator 600 includes a housing 620 with an inlet 622 for receiving breathing gas. The housing 620 may include a cover 640, which may be removable. The cover 640 includes an outlet 642 in fluid communication with the inlet 622. FIG. 2 depicts an embodiment with a cylindrical housing. A shutter 630 with a first orifice 632 is disposed in the path between the inlet 622 and the outlet 642. The shutter 630 is configured such that as the shutter rotates, the first orifice 632 is intermittently aligned between the inlet and the outlet allowing gas to flow from the inlet to the outlet. The shutter may be completely or partially contained within the housing.

The valve 600 may include a motor 660 coupled to the shutter 630. The motor may be configured to rotate the shutter. The motor may be any type of motor known in the art. For example, an electric motor, a pneumatic motor, etc. The motor may be directly coupled to the shutter, for example, by way of an axle sealed through the housing drives. The motor may drive the shutter at a constant angular velocity. The rotational period determines the respiratory rate (breaths per minute). In some embodiments, the valve includes a crank (or a coupler for connection to a crank) for manual rotation of the shutter. In this way, the shutter may be rotated “by hand” or other manual operation. Such a crank or coupler may be present in addition to or as an alternative to a motor.

In some embodiments, an exhaust pressure regulator is connected to the outlet. The exhaust pressure regulator can be used to control a positive end-of-expiration pressure (“PEEP”).

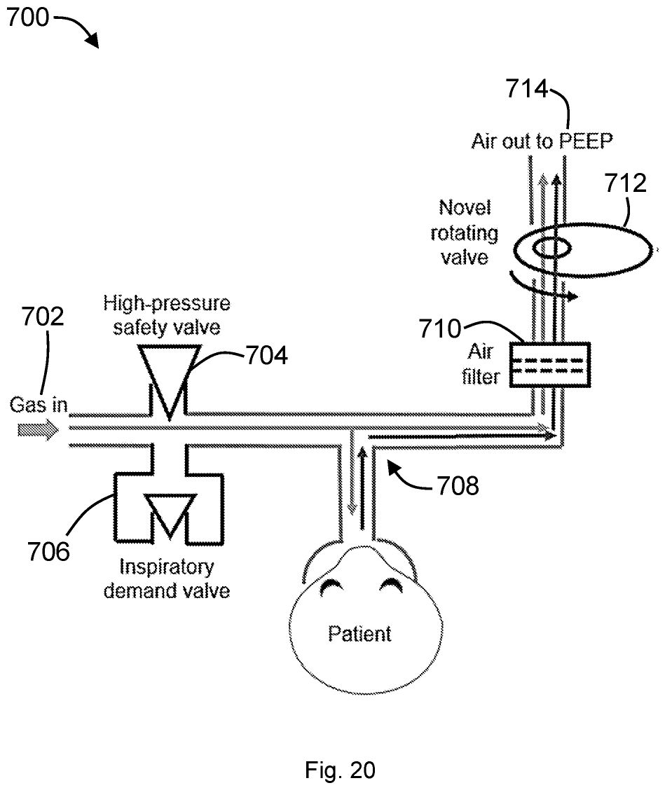

FIG. 20 shows a schematic of a ventilator system 700 using the above-described ventilator valve 712. Gas suitable for ventilating an individual comes in from a supply 702 at pressure greater than or equal to P_{high}. Before reaching the individual, the gas circuit passes by a high-pressure safety valve 704 set to release if the supply pressure exceeds P_{high} in order to prevent over-pressurizing the lungs of the individual. The circuit may also include an inspiratory demand valve 706 configured to open to the atmosphere if the individual makes a spontaneous inspiratory effort sufficient to decrease the circuit pressure below a set threshold. This allows the individual to increase their minute ventilation when sufficiently motivated to do so. The circuit then reaches a Y-junction 708 via a short segment (a few cm in length) to the endotracheal tube. Inspiratory gas takes this pathway when the lungs are being inflated. When the individual expires, the gas from the lungs traverses this pathway in the opposite direction until it reaches the Y-junction 708. At this point, continued pressure from the gas source forces the expired gas to exit along the expiratory circuit (to the right in the diagram) where it passes through a conventional medical respiratory filter 710 before exiting via the rotary valve 712 that controls T_{high} and T_{low} as described in FIG. 18. The exiting gas may pass through a positive end-expiratory pressure (PEEP) valve 714 if it is desired that T_{low} be greater than 0 (all pressures are expressed relative to atmospheric pressure). The expired gas can either be vented to the room or further processed as desired.

As the shutter rotates, the individual's lungs will be exposed to pressure P_{high} for duration T_{high} (while the shutter orifice is not aligned between the inlet and the outlet) and pressure P_{low} for duration T_{low} (while the shutter is aligned between the inlet and the outlet) throughout a single rotation. Gas from the individual's lungs exits through the valve when a radially-aligned orifice in the rotating shutter is coincident with the inlet and outlet orifices that face each other in the sides of the housing. When the shutter orifice rotates past these outer orifices (inlet and outlet), the valve is closed and expiration cannot take place. The duration of inspiration versus expiration is given by the angle of rotation of the disk through which the inner and outer holes overlap as a fraction of 360 degrees.

The inlet, the outlet, and the orifice of the shutter may have the same size and shape as each other, or one or more the inlet, the outlet, and the orifice may have a different size and/or shape as the others. By modifying the size and/or shape of these openings, the pressure and flow characteristics of a ventilator may be modified. For example, by modifying the size, the ratio of T_{high} to T_{low} can be altered. In another example, altering the shape of the orifice with respect to the inlet may allow modification of the slope of the pressure curve in FIG. 1 (e.g., more or less gradual change in pressure, etc.) In some embodiments, the shutter orifice is an arc-shaped orifice and the inlet and/or outlet are similarly shaped, but wider, arc-shaped orifices that face each other in the sides of the housing.

The rotational speed of the motor may be increased or decreased as desired for a particular individual. In an exemplary embodiment with triangular openings (inlet, outlet, and/or orifice) the angles subtended by the triangular openings may at least partially determine T_{low}. Adjustments in this angle, such as provided by a rotating shutter, would allow T_{low} to be adjusted independently of the breathing rate.

This system requires a source of gas at a constant pressure, P≥P_{high}, with the flow capacity necessary to meet the ventilatory needs of the individual and to deliver APRV with suitable lung fill rates following each expiration (as will be determined by the medical professional for each ventilated individual). The gas may be of any composition (oxygen partial pressure, humidity, etc.) selected by an operator and/or the provider of the gas source.

The system can be extended to include a second rotary valve through which the inspiratory gas passes, and in which the overlapping orifices are arranged so that inspiration is precisely out of phase with inspiration.

This system requires a source of gas at a constant pressure, P, and flow capacity necessary to provide ventilation APRV. P should probably be at least 40 cmH₂O (the maximum P_{high}), and the flow capacity should such as required to quickly inflate the lungs at this pressure. The composition of the gas (oxygen partial pressure, humidity, etc.) is up to the provider of the gas source.

It should be noted that the use of “abut” in describing components does not necessarily require a sealing (e.g., air tight, etc.) or other contacting relationship between such components. In some embodiments, abutting components may simply be sufficiently proximate such to accomplish the functions of such components as will be apparent to one skilled in the art. In some embodiments, abutting components may be in at least partial contact with one another. In some embodiments, abutting components may be in sealing relationship (at the working pressures) with one another. A component may abut another component directly or indirectly. For example, in some embodiments, a gasket, O-ring, seal (e.g., brush seal, etc.), adhesive, lubricant, wear strip, filler, or other element or combination of elements may be provided between the abutting components.

In some embodiments, each orifice is the same size as the corresponding fixed port. In some embodiments, one or both orifices may have a different size (larger, smaller, or both) from the corresponding fixed ports. The first orifice may be the same size and/or shape or a different size and/or shape from the second orifice. The first fixed port may be the same size and/or shape or a different size and/or shape from the second fixed port. Embodiments of the disclosure are not to be limited to only those shapes of “holes” (e.g., orifices, stationary orifices, slots, fixed ports, etc.) disclosed herein. Such holes may be the same or different size as any of the other holes of a particular embodiment. Such holes may be the same or different shape as any of the other holes of a particular embodiment. For example, a wedge shaped first orifice may interface with a circular first stationary orifice. The shapes may be regular, irregular, symmetrical, asymmetrical, or otherwise.

In another aspect of the present disclosure, a method for ventilation is provided. For example, a method may comprise providing a patent exhaust gas pathway to a patient thereby allowing expiration through the exhaust gas pathway. The method includes rotating a shutter so as to occlude the exhaust gas pathway. In some embodiments, rotation of the shutter also opens (at least partially) an inspiratory gas pathway through which breathing gas is provided. The shutter is then rotated to at least partially open the exhaust gas pathway. The direction of rotation to occlude the exhaust gas pathway may be the same as the direction of rotation to open the exhaust gas pathway. In other embodiments, the direction of rotation to occlude the exhaust gas pathway is different from the direction of rotation to open the exhaust gas pathway. In embodiments where an inspiratory gas pathway is present, rotating the shutter to at least partially open the exhaust gas pathway will cause the inspiratory gas pathway to be occluded by the shutter.

### “Vermontilator” Example

The following is a non-limiting exemplary ventilator according to an embodiment of the present disclosure. The example is intended only to illustrate an embodiment and is not limiting in any way.

### Functional and Operational Characteristics

The Vermontilator Model EA2020.1 is a low-cost, easy-to-produce mechanical ventilator designed to respond to the possibility that hospital resources will be seriously overwhelmed when the number of COVID-19 patients in respiratory failure reaches its peak. The Vermontilator provides the mode of ventilation known as APRV (Airway Pressure Release Ventilation) because there is strong evidence that this mode of ventilation may be beneficial for patients with acute respiratory distress syndrome (ARDS) caused by COVID-19.

### Ventilation Circuit Configuration

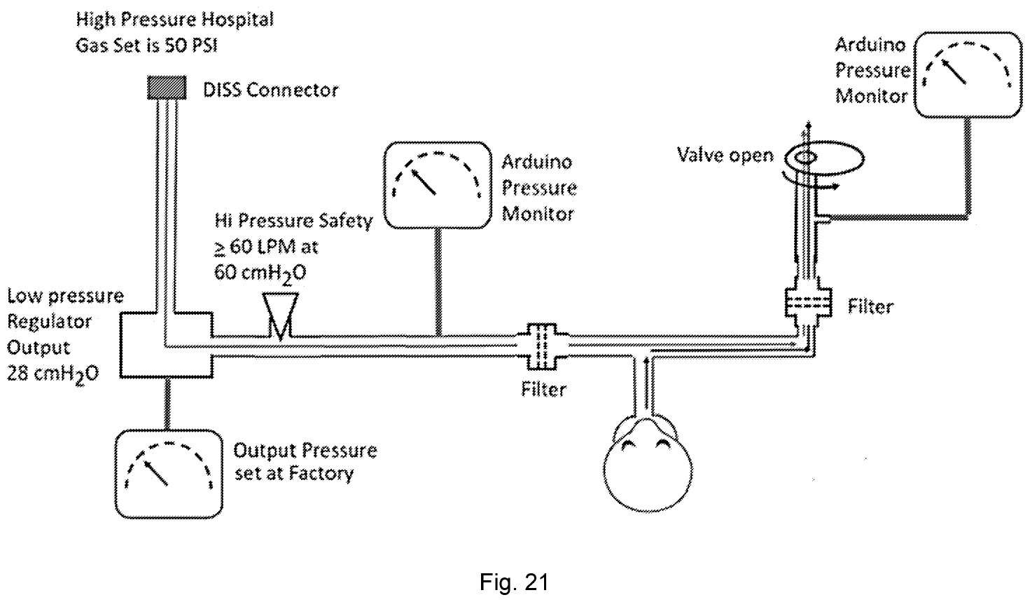

FIG. 21 shows a schematic of the functional components of the system. Gas suitable for ventilating the patient comes in from a supply at a pressure of 50 psi. This passes through a regulator that outputs a pressure of P_{high}=28 cmH₂O with a flow capacity greater than 50 liters/min. Protection against over-pressurization of the patient is provided by a passive, non-adjustable high pressure safety value that opens at a factory-set pressure of 60 cmH₂O and that can pass more than 60 liters/min when open. If this valve opens due to patient coughing, resetting it is quickly achieved by pushing a button on the back of the unit.

### Component Characteristics

The regulated gas source connects to the inspiratory arm of standard ventilator Y-tubing. The inspiratory and expiratory arms to the Y-tubing meet at the junction of Y, which leads via a short segment to the endotracheal tube. A rotating valve (shutter) controls the timing of inspiration and expiration. When the valve is positioned so that the expiratory pathway is blocked, a pressure of P_{high} is applied to the patient's lung. This causes gas to flow into the lungs until the elastic recoil pressure of the respiratory system reaches P_{high}. When the valve rotates to a position that opens the expiratory pathway, a pressure of P_{low}=0 is applied to the patient's lungs. This allows gas to escape from the lungs.

An electronic monitoring system uses an Arduino to monitor the cyclically changing pressures within the ventilator circuit. If these pressure signals fall outside a designated range of behavior patterns an alarm will sound. This signals either a malfunction of the Vermontilator or a leak in the circuit.

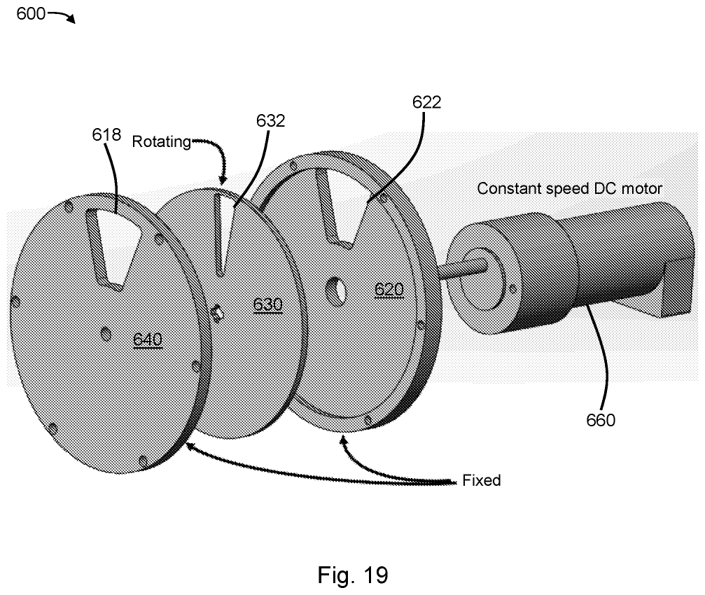

FIG. 19 shows the valve design that causes the patient's lungs to be exposed to pressure P_{high} for duration T_{high}, and pressure P_{low} for duration T_{low} throughout a single rotation. The Rotating Valve includes an inner disk that rotates flush against the inside of the outer casing. Gas from the patient's lungs exits through the valve when a radially-aligned arc-shaped orifice in the rotating disk is coincident with similar, but wider, arc-shaped orifices (stationary orifices) that face each other in the circular sides of the casing. When the inner disk rotates past these outer orifices, the valve is closed and expiration cannot take place. The ratio of expiratory duration to inspiratory duration is given by the angle of rotation of the disk through which the inner and outer holes overlap as a fraction of 360 degrees. A basic rotary motor with an axle sealed through the center of the housing drives the rotating disk at a constant angular velocity. The rotation period determines the respiratory rate (breaths per minute).

A pressure relief valve is positioned just downstream from the main pressure regulator on the inspiratory circuit of the air path to the patient (see FIG. 21). The pressure relief valve acts to prevent high pressure resulting from component failure upstream from reaching the patient's lungs.

### Setup

A cycle time switch on the front panel of the ventilator adjusts the breath period to be either 4.4 sec, 5.5 sec, or 6.6 sec. Expiratory duration for each breath period is 0.4 sec, 0.5 sec, and 0.6 sec, respectively. Gas ports for entry and exit are provided. Gas provided through such ports can have an oxygen concentration from that of room air to 100%, corresponding to an FiO₂ of 0.21-1.0.

The expiratory pathway through the Vermontilator unit is downstream of a respiratory filter attached to the ventilator tubing, so this pathway is not expected to become contaminated by exhaled gas and secretions from the patient. In any case, gas passes through this pathway only after exiting the patient's lungs, so cross-contamination will not occur between patients. The inspiratory pathway normally conveys only fresh gas from the gas source. However, if the patient exhales forcibly or coughs enough to raise the pressure in the circuit above 60 cmH₂O, a safety pressure release valve will open. This may allow exhaled gas and secretions to pass retrogradely along the inspiratory pathway. Cleaning and disinfecting this pathway between patient use is simply a matter of removing the connections to the pathway and swabbing it through with a suitable disinfecting agent.

The patient's breath may condense in the ventilator tubing during use, as can occur with any mechanical ventilator that uses such tubing. Keeping the distal openings of the inspiratory and expiratory arms of the Y-tubing below the level of the endotracheal tube opening will ensure that condensates do not run back into the trachea. The suction port facing the endotracheal tube in the Y-tubing allows a suction catheter to be inserted into the lungs for the clearance of secretions.

Although the present disclosure has been described with respect to one or more particular embodiments, it will be understood that other embodiments of the present disclosure may be made without departing from the spirit and scope of the present disclosure.

1. A ventilator, comprising:
a housing having a first fixed port;
a shutter configured to cooperate with the housing to enclose an interior of the housing, the shutter being rotatable about an axis of rotation and having a first orifice at a first radial distance from the axis of rotation;
a stationary plate abutting the shutter, the stationary plate having a first stationary orifice; and
wherein the first orifice of the shutter is configured to at least partially align with the first stationary orifice of the stationary plate over a first rotational distance of the shutter thereby forming a first variable port.

2. The ventilator of claim 1, wherein the stationary plate has a second stationary orifice and the shutter has a second orifice configured to at least partially align with the second stationary orifice over a second rotational distance of the shutter, thereby forming a second variable port.

3. The ventilator of claim 2, wherein the second orifice is at a second radial distance from the axis of rotation which is greater than the first radial distance of the first orifice.

4. The ventilator of claim 2, wherein the housing includes a separator configured to divide the interior of the housing into an inner chamber and an outer chamber, wherein the first fixed port and the first variable port are located on the inner chamber and the second fixed port and the second variable port are located on the outer chamber.

5. The ventilator of claim 2, further comprising an adjustment plate abutting the stationary plate, the adjustment plate having a first adjustment slot configured to at least partially align with the first stationary orifice of the stationary plate.

6. The ventilator of claim 5, wherein the adjustment plate further comprises a second adjustment slot configured to at least partially align with the second stationary orifice of the stationary plate.

7. The ventilator of claim 5, wherein the adjustment plate is configured to be rotated to vary an alignment of the first adjustment slot with the first stationary orifice.

8. The ventilator of claim 6, wherein each of the first adjustment slot and the second adjustment slot further includes a wiper configured to extend through the respective first or second stationary orifice of the stationary plate to contact the shutter.

9. The ventilator of claim 5, further comprising a first duct disposed on the adjustment plate and sized to interface with the first adjustment slot of the adjustment plate.

10. The ventilator of claim 1, further comprising a motor configured to rotate the shutter relative to the housing.

11. The ventilator of claim 1, further comprising a crank configured to rotate the shutter when turned by an operator.

12. The ventilator of claim 1, wherein the shutter is contained with the housing.

13. The ventilator of claim 1, wherein the shutter is configured to rotate reciprocally between two angular positions.

14. A ventilator valve, comprising:
a housing, the housing having an inlet for receiving breathing gas, the housing having a cover with an outlet in fluid communication with the inlet; and
a shutter rotatably disposed in a path between the inlet and the outlet, the shutter having an orifice configured to be in intermittent alignment with the inlet and the outlet such that, as the shutter is rotated, the inlet and outlet are occluded when the orifice is not aligned and patent when the orifice is at least partially aligned between the inlet and the outlet.

15. The ventilator valve of claim 14, further comprising an exhaust pressure regulator for controlling a positive-end-of-expiration pressure (“PEEP”) and connected to the outlet of the housing.

16. The ventilator valve of claim 14, further comprising a coupler configured for connection to a crank for manually rotating the shutter.

17. The ventilator valve of claim 14, wherein the shutter is a disk.

18. The ventilator valve of claim 14, wherein the housing is cylindrical.

19. The ventilator valve of claim 14, wherein the shutter is contained within the housing.

20. A ventilator, comprising:
a Y-shaped conduit having an inspiratory leg configured to be connected to a source of breathing gas, a patient leg configured to be connected to an endotracheal tube, and an expiratory leg;
a safety valve coupled to the inspiratory leg; and
a first ventilator valve according to claim 14 and connected to the expiratory leg.

21. The ventilator of claim 20, further comprising an inspiratory demand valve connected to the inspiratory leg.

22. The ventilator of claim 20, further comprising a filter coupled in the expiratory leg such that gas flowing through the expiratory leg is filtered.

23. The ventilator of claim 20, further comprising a second ventilator valve, the second ventilator valve comprising:
a housing, the housing having an inlet for receiving breathing gas, the housing having a cover with an outlet in fluid communication with the inlet; and
a shutter rotatably disposed in a path between the inlet and the outlet, the shutter having an orifice configured to be in intermittent alignment with the inlet and the outlet such that, as the shutter is rotated, the inlet and outlet are occluded when the orifice is not aligned and patent when the orifice is at least partially aligned between the inlet and the outlet,
and connected to the inspiratory leg, the second ventilator valve configured to be synchronized with the first ventilator valve such that gas flow through the inspiratory leg is out of phase with gas flow through the expiratory leg.
